(19) United States
(12) Patent Application Publication
(10) Pub. No.: US 2026/0207374 A1
(43) Pub. Date: Jul. 23, 2026

(54) INTRAUTERINE DEVICE (IUD) SYSTEM

(71) Applicant: 3DAUGHTERS, INC., Fort Lauderdale, FL (US)

(72) Inventors: Paul James Mulhauser, New York, NY (US); Mika Becktor, Coral Gables, FL (US)

(21) Appl. No.: 19/144,237

(22) PCT Filed: Dec. 29, 2023
(86) PCT No.: PCT/US2023/086472

§ 371 Date: Jun. 27, 2025

Related U.S. Application Data
(60) Provisional application No. 63/436,341, filed on Dec. 30, 2022.
(60) Provisional application No. 63/541,557, filed on Sep. 29, 2023.

(51) Int. Cl. A61F 6/14; A61F 6/18

(52) U.S. Cl. CPC A61F 6/142; A61F 6/18

(57) ABSTRACT

An intrauterine device (IUD) system is disclosed. The IUD system includes a plurality of elements, each element having a length of 10 mm or less. Each element includes a magnetic core and a coating exterior to the magnetic core, the magnetic core being dimensioned to assemble the plurality of elements into a conformation that resists expulsion from a uterus of a subject. A method of providing controlled contraception or treating or preventing a uterine disease, condition, or symptom thereof in a subject is also disclosed. The method includes delivering to a uterus of the subject the plurality of elements. A method of facilitating controlled contraception or treating or preventing a uterine disease, condition, or symptom thereof in a subject is also disclosed. The method includes providing the plurality of elements. A kit including a plurality of elements and a delivery device is also disclosed.

### CROSS REFERENCE TO RELATED APPLICATIONS

This application claims priority under 35 U.S.C. § 119(e) to U.S. Provisional Application Ser. No. 63/436,341 titled “INTRAUTERINE DEVICE (IUD) SYSTEM” having a filing date of Dec. 30, 2022 and U.S. Provisional Application Ser. No. 63/541,557 titled “INTRAUTERINE DEVICE (IUD) SYSTEM” having a filing date of Sep. 29, 2023, each of which is incorporated herein by reference in its entirety for all purposes.

### FIELD OF TECHNOLOGY

Aspects and embodiments disclosed herein are directed toward intrauterine device (IUD) systems and methods of providing contraception and/or treatment or prevention of a uterine disease, condition, or symptom thereof. In particular, aspects and embodiments disclosed herein are directed toward IUD systems having a plurality of magnetic IUD elements.

### SUMMARY

In accordance with one aspect, there is provided an intrauterine device (IUD) system. The IUD system may include a plurality of elements. Each element may have a length of 10 mm or less. Each element may comprise a magnetic core and a coating exterior to the magnetic core. The magnetic core may be dimensioned to assemble the plurality of elements into a conformation that resists expulsion from a uterus of a subject.

In some embodiments, the IUD system includes at least three elements.

In some embodiments, the IUD system includes three elements. Each element may have the magnetic core dimensioned to assemble the plurality of elements into a triad conformation.

In some embodiments, each element comprises a shell exterior to the magnetic core. In some embodiments, the IUD system includes three elements, each element having the shell dimensioned to assemble the plurality of elements into a triad conformation.

In some embodiments, the shell has an average thickness between 0.10 mm and 1.50 mm.

In some embodiments, the coating is an anti-fertility agent coating and the anti-fertility agent coating is embedded in the shell.

In some embodiments, the magnetic core is an axially charged magnetic core.

In some embodiments, the magnetic core is dimensioned to de-assemble the plurality of elements into an axial or generally aligned conformation that allows retrieval from the uterus of the subject with a magnetic retrieval device.

In some embodiments, each element consists of the magnetic core and the coating.

In some embodiments, each element has a rounded cross-sectional area having a diameter between 2.0 mm and 6.0 mm.

In some embodiments, each element is dimensioned to pass through the cervix of the subject.

In some embodiments, each element is dimensioned to fit within a 4.0 mm to 5.0 mm OD catheter.

In some embodiments, each element has a length between 2.0 mm and 10 mm.

In some embodiments, the magnetic core has rounded or beveled edges.

In some embodiments, the coating is an anti-fertility agent coating and the anti-fertility agent comprises a cytotoxic agent.

In some embodiments, the cytotoxic agent coating comprises copper.

In some embodiments, the anti-fertility agent coating has a copper surface area of between 175 mm² and 380 mm².

In some embodiments, the coating is an anti-fertility agent coating and the anti-fertility agent comprises a hormonal agent.

In some embodiments, the coating is substantially free of a hormonal agent.

In some embodiments, the coating is substantially free of nickel and/or nitinol.

In some embodiments, the magnetic core includes a south pole and a north pole opposite the south pole.

In some embodiments, the magnetic core forms at least 50% of a volume of each element.

In some embodiments, each of the plurality of elements are structurally independent from one another.

In accordance with another aspect, there is provided a method of providing controlled contraception or treating or preventing a uterine disease, condition, or symptom thereof in a subject. The method may comprise delivering to a uterus of the subject a plurality of elements, each element having a length of 10 mm or less, comprising a magnetic core and a coating exterior to the magnetic core, the magnetic core being dimensioned to assemble the plurality of elements into a conformation that resists expulsion from the uterus of the subject.

In some embodiments, the method may comprise delivering each element individually.

In some embodiments, the method may comprise delivering each element sequentially and/or simultaneously.

In accordance with another aspect, there is provided a kit comprising an intrauterine device (IUD) system including a plurality of elements, each element having a length of 10 mm or less, comprising a magnetic core and a coating exterior to the magnetic core; a delivery device dimensioned to allow placement of the plurality of elements in the uterus of a subject; and instructions to deliver the plurality of elements to the uterus of the subject using the delivery device.

In some embodiments, the kit may further comprise a retrieval device dimensioned to allow retrieval of the plurality of elements from the uterus of the subject.

In accordance with another aspect, there is provided a method of facilitating controlled contraception or treating or preventing a uterine disease, condition, or symptom thereof in a subject. The method may comprise providing a plurality of elements, each element having a length of 10 mm or less, comprising a magnetic core and a coating exterior to the magnetic core, the magnetic core being dimensioned to assemble the plurality of elements into a conformation that resists expulsion from the uterus of a subject. The method may comprise providing instructions to deliver the plurality of elements to the uterus of the subject.

The disclosure contemplates all combinations of any one or more of the foregoing aspects and/or embodiments, as well as combinations with any one or more of the embodiments set forth in the detailed description and any examples.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a schematic drawing of a uterine implant element, according to one embodiment;

FIG. 1B is a schematic drawing of three uterine implant elements arranged in a triad conformation, including a sectional and a partial sectional view of the uterine implant elements, according to one embodiment;

FIG. 1C is a sectional drawing of three uterine implant elements arranged in a triad conformation, showing shell thickness relationships, according to one embodiment;

FIG. 1D is a sectional drawing of a uterine implant element, showing beveled features, according to one embodiment;

FIG. 1E is a sectional drawing of three uterine implant elements arranged in a triad configuration, showing beveled conjoining relationships, according to one embodiment;

FIGS. 2A-2C are sectional views of varied uterine implant elements, according to certain embodiments;

FIG. 3A is a schematic drawing showing magnetic attraction of three uterine implant elements arranged in a triad configuration, with each element containing a cylindrical magnetic core, according to one embodiment;

FIG. 3B is a schematic drawing showing magnetic attraction of three uterine implant elements arranged in a triad configuration, with each element containing a cylindrical magnetic core with beveled edges, according to one embodiment;

FIG. 3C is a schematic drawing showing overall profile of three uterine implant elements arranged in a triad configuration, according to one embodiment;

FIG. 3D is a schematic drawing showing overall profile of three uterine implant elements arranged in a triad configuration, having a smaller overall profile than the uterine implant elements of FIG. 3C, according to one embodiment;

FIG. 4 is a sectional view of a uterine implant element, according to one embodiment; FIG. 5 is a chart comparing magnetic force for several magnetic core geometries and assembly configurations, according to certain embodiments;

FIG. 6 is a schematic drawing of a uterine implant element, according to one embodiment;

FIG. 7 is a sectional view of a uterine implant element, according to one embodiment;

FIG. 8 is a sectional drawing of a uterine implant element, showing magnetic flux patterns of an axial charge, according to one embodiment;

FIG. 9 is a schematic drawing of an IUD system, in accordance with one embodiment;

FIG. 10 is a partial sectional view of an IUD system, showing magnetic flux patterns, in accordance with one embodiment; and

FIG. 11 is a schematic drawing of a uterine implant element, in accordance with one embodiment.

### DETAILED DESCRIPTION

Contraception may be provided by a variety of methods. One of the safest and most effective methods is the placement of an intrauterine device (IUD) in the uterus of the subject to prevent pregnancy as a result of sexual intercourse. The IUD will generally have a contraceptive effect for the duration of the stay in the uterus. The contraceptive effect may be easily reversed by removing the IUD from the uterus.

Current IUDs on the market are T-shaped framed devices made of plastic that release a spermicide or a hormonal agent to provide contraception. These conventional IUDs may be very effective, but the T-shaped frame design leads to significant pain upon insertion, perforation of the uterus, expulsion from the uterus, and vaginitis caused by an external retrieval string. Accordingly, there is a need for improved IUDs that provide easier and less painful insertion.

The embodiments described herein provide an intrauterine device (IUD) system, e.g., a frameless IUD system, including one or more uterine implant elements which provide high effectiveness, are easily implantable, easily retrievable, and exhibit a high retention rate, without affecting future fertility. In certain embodiments, the IUD system is free of an external retrieval string.

### Select Definitions

As used herein, “anti-fertility agent” means a drug, compound, or biological product having a contraceptive effect on a subject. The anti-fertility agent may be an active agent of a composition. In some embodiments, the anti-fertility agent may be a hormonal agent. In some embodiments, the anti-fertility agent may be a cytotoxic agent, sometimes referred to as a “spermicide”. The anti-fertility agent may also be referred to as a “contraceptive agent” herein.

As used herein, “hormonal agent” means an anti-fertility agent comprising a hormone or signaling molecule. The hormonal agent may be a naturally occurring hormone, e.g., progesterone. The hormonal agent may be a synthetic hormone, e.g., progestogens such as progestin.

As used herein, “cytotoxic” agent means a non-hormonal anti-fertility agent that targets sperm cells and/or ova cells. The cytotoxic agent may also be referred to as a spermicide or spermicidal agent. In certain exemplary embodiments, the cytotoxic agent may be copper.

As used herein, “contraception” means the prevention of pregnancy as a consequence of sexual intercourse. Contraception may include barrier methods, preventing ovulation in the female, inactivating or killing sperm, inhibiting or preventing motility of sperm, modulating cervical mucus, and/or preventing the fertilized ovum from implanting in the uterus.

As used herein, “inert” means non-toxic or non-harmful to surrounding tissues. An inert material may be biocompatible.

As used herein, a “subject” may include an animal, a mammal, a human, a non-human animal. The term “subject” is intended to include human and non-human animals, for example, mammals, large animals, livestock animals, companion animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the disclosure includes all mammals, such as non-human primates, domesticated, laboratory/research, and agriculturally useful animals, for example, horse, sheep, goat, dog, cat, cow, pig, rat; wild carnivores, such as wolf, bear, cheetah, tiger, leopard, lion; ungulates such as wild hogs, boars; or ruminants/pseudo ruminants such as camels, deer, antelope, buffaloes; or large mammals such as elephants; among others. The subject may be biologically equipped to bear offspring. The subject may have a uterus or uterine cavity. The subject may be female. The subject may be of a childbearing age.

As used herein, “retention rate” means a percentage of IUD systems that remain in uterus of the subject after a selected period of time has elapsed from the date of insertion without voluntary removal. The elapsed period of time can be 24 hours, 48 hours, 72 hours, 7 days, one month, three months, six months, 9 months, 12 months, 18 months, 24 months, 36 months, 48 months, 60 months, 72 months, 84 months, 96 months, 108 months, 120 months, 132 months, 144 months, or more.

As used herein, treatment of a disease or condition refers to reducing the severity or frequency of at least one symptom of that disease or condition, compared to a similar but untreated patient. Treatment can also refer to halting, slowing, or reversing the progression of a disease or condition, compared to a similar but untreated patient. Treatment may comprise addressing the root cause of the disease and/or one or more symptoms.

### Intrauterine Device (IUD) System

The disclosure provides an intrauterine device (IUD) system. The system may have one or more uterine implant elements, also referred to as intrauterine device elements, “IUD elements” or “elements” herein. The element may be a device for insertion into the uterus of a subject to provide contraceptive effects, inhibit, limit, or reduce side effects of menstrual periods, e.g., discomfort, pain, excessive bleeding, cramps, or inflammation, and/or treat or prevent uterine diseases, conditions, and symptoms thereof, e.g., abnormal uterine bleeding, irregular menstrual cycle, irregular vaginal discharge, endometriosis, adenomyosis, uterine fibroids, polyps, or cysts, polycystic ovary syndrome (PCOS), pelvic inflammatory disease, endometrial hyperplasia, uterine cancer, dysuria, genital tuberculosis, uterine prolapse, bacterial, parasitic, or viral sexually transmitted diseases, discomfort, pain, cramps, or inflammation.

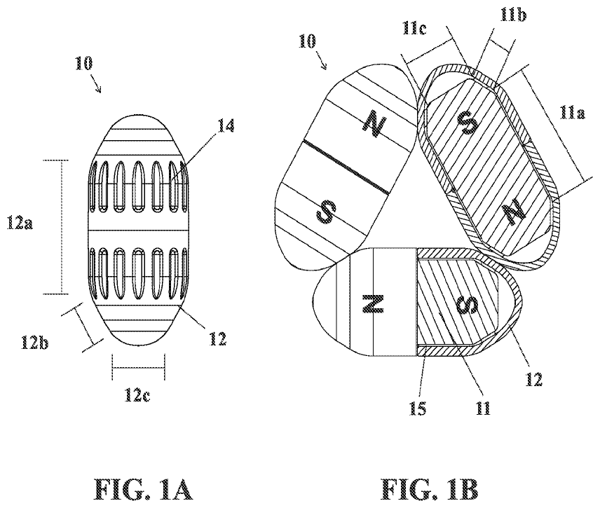

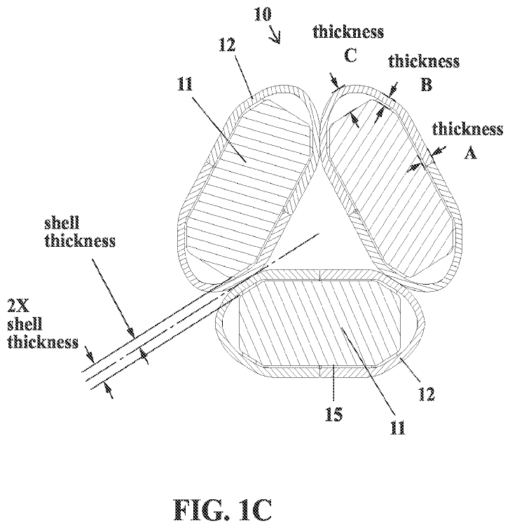

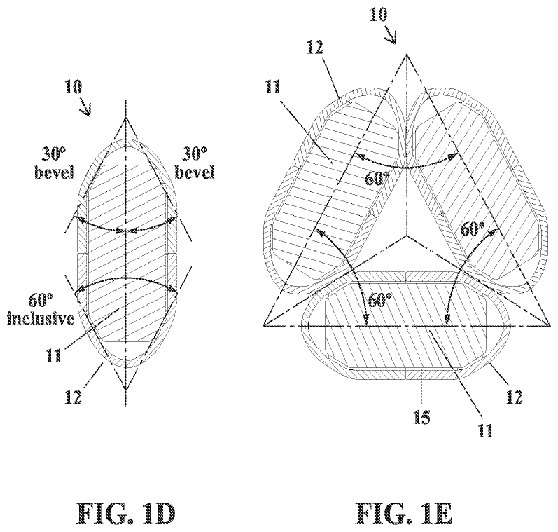

Exemplary elements are shown in FIGS. 1A-1B. FIG. 1A shows a lateral side view of one uterine implant element 10. FIG. 1B shows a lateral side view of several uterine implant elements 10 assembled in a conformation, in which one element is shown in a sectional view and another element is shown in a partial sectional view. The elements may include a magnetic core 11. The magnetic core may be an axially charged magnetic core, with opposing north (N) and south(S) poles at each end.

The element 10 may comprise one or more layers exterior to the magnetic core 11. The element 10 may comprise a shell 12 exterior to the magnetic core 11. In other embodiments, the element 10 may be substantially free of the shell 12. In one particular embodiment, the element 10 may comprise a coating exterior to the shell 12. In other embodiments, the element 10 may be substantially free of a coating exterior to the shell 12. The one or more layers of the element 10 may be selected based on function or manufacture.

The contraceptive or therapeutic effect of the elements may be provided by implantation of the elements in the uterus. The contraceptive or therapeutic effect may be provided by the geometry and physical features of the elements and/or arrangement or configuration of elements when placed in the uterus of the subject. In certain embodiments, the elements may be dimensioned to provide contraception. For example, in certain veterinary applications, such as equine mares, the suppression of estrus provided by the presence and/or geometry of the elements may provide contraception. In certain embodiments, the elements may be dimensioned to provide treatment or prevention of a uterine disease, condition, or symptom thereof. A therapeutic effect may be provided in addition to or alternatively to the contraceptive effect.

In some embodiments, the contraceptive effect of the elements may be provided by an anti-fertility agent. In one particular embodiment, the anti-fertility agent may be provided in a coating exterior to the magnetic core 11. In certain embodiments, the shell 12 may be formed of or comprise the anti-fertility agent. For instance, the anti-fertility agent may be homogeneously or non-homogeneously embedded in the shell 12. In other embodiments, the anti-fertility agent coating may be exterior to the shell 12. Thus, the anti-fertility agent coating may form an outermost layer of the element 10, such as an encasement, wire, sheath, or other structure on an exterior surface of the element 10.

The coating, e.g., anti-fertility agent coating or other coating, need not cover the complete surface of the element 10, for instance, the coating may cover at least 25%, 25% 50%, 50%-75%, or 75%-100% of the exterior surface of the element 10. In yet other embodiments, the anti-fertility agent coating may form an internal layer of the element 10 (for example, between the core 11 and shell 12 layer) and be released through the shell 12. Exemplary anti-fertility agents include hormonal agents and cytotoxic agents.

The element may be dimensioned to be ergonomic (compatible with uterine tissues, optionally, maximize effectiveness, efficiency, safety, and comfort when inserted within the uterus of the subject). In some embodiments, the element may be ergonomic by adapting to the uterine shape and environment. The core and/or the shell of the element may be dimensioned to be ergonomic, providing the desired properties of the element. The element, e.g., core and/or shell, may have smooth, oval, or rounded edges. The element, e.g., core and/or shell, may be dimensioned to prohibit, limit, or reduce distortion or perforation of the uterine lining. The element, e.g., core and/or shell, may be dimensioned to reduce the likelihood of side effects associated with insertion, e.g., discomfort, pain, bleeding (e.g., excessive bleeding), cramps, or inflammation. In some embodiments, the element is oval (for example, having a rounded and slightly elongated outline or shape, like that of an egg; elliptical; ellipsoidal configuration).

Each element 10 may have a length between about 1 mm to about 100 mm, for example, between about 1 mm to 50 mm, 1 mm to 30 mm, 1 mm to 20 mm, 1 mm to 10 mm, or 5 mm to 10 mm. In exemplary embodiments, for example, for use in humans, the element may have a length of less than 10 mm.

Each element 10 may have a width of between about 1 mm to about 30 mm, for example, between about 1 mm to 15 mm, 1 mm to 10 mm, or 2 mm to 6 mm. In exemplary embodiments, for example, for use in humans, the element may have a width capable of passing through the human cervix for placement within the uterine cavity. The exemplary element may have a width between 3 mm and 4.5 mm. In some embodiments, the element may have a width capable of passing through a catheter for implantation in the uterine cavity. The exemplary element may have a width of less than 4.5 mm, for example, 3 mm to 4.4 mm, 3 mm to 4.2 mm, or 3 mm to 4.0 mm.

Each element may have a mass of between 0.1 g to 100 g, for example, 0.1 g to 1.0 g, 0.1 g to 0.5 g, 0.4 g to 0.6 g, 0.6 g to 0.8 g, 0.5 g to 1.0 g, 0.5 g to 50 g, 0.5 g to 10 g, 0.5 g to 5 g, or 1 g to 2 g. In exemplary embodiments, each element may have a mass of about 0.5 g, 0.4 g to 0.6 g, or 0.6 g to 0.8 g. Together, three elements may have a mass of about 1.2 g, 1.5 g, 1.8 g, 2.1 g, or 2.4 g. In particular, exemplary elements having a polymeric shell may have a mass of about 0.1 to 0.3 g. Together, three elements having a polymeric shell may have a mass of about 0.6 g to 0.8 g.

The elements may be dimensioned to fit the target subject, for example, to pass through a cervix of the target subject and fit within a uterine cavity of the target subject. As disclosed herein, the uterine cavity may refer to the space inside the uterus, within opposed anterior and posterior moist mucous endometrial membranes and myometrial muscles. In certain embodiments, the elements 10 may be dimensioned to fit primates. For primates (e.g., human), the length of the element may be about 1 mm to about 10 mm, for example, about 7 mm to about 8.5 mm, and the width (or diameter) may be about 2 mm to about 6 mm, for example, about 3 mm to about 3.5 mm, about 3.5 mm to about 4 mm, or about 4 mm to about 5 mm.

The magnetic core 11 of the element 10 may be formed of or comprise a magnetic material. Exemplary magnetic materials include iron (or an ore, alloy, or other material) that has its component atoms so ordered that the material exhibits properties of magnetism, such as attracting other iron-containing objects or aligning itself in an external magnetic field. For example, the core 11 may be formed of or comprise materials that are magnetized (called ferromagnetic or ferrimagnetic). Exemplary magnetic materials include iron, nickel, cobalt, or an alloy thereof. The core may comprise an alloy of rare earth metal (e.g., neodymium, e.g., highly attractive neodymium), and a naturally occurring mineral, such as lodestone. In some embodiments, the elements may be substantially free of nickel.

The element 10 may comprise an optional shell 12 exterior to the magnetic core 11. The shell 12 may be formed of an inert material. The shell 12 may protect the magnetic core 11 from structural damage, such as, fracturing, cracking, chipping, corrosion, or any other structural damage. For example, neodymium cores may tend to be brittle. Multiple neodymium cores may fracture if impacted together. A shell can protect the neodymium core to prevent fracturing.

The shell 12 may have an average thickness of 0.1 mm to 1.5 mm, for example, 0.1 mm to 0.15 mm, 0.15 mm to 0.25 mm, 0.25 mm to 0.5 mm, 0.1 mm to 0.5 mm, or 0.5 mm to 1.5 mm. In certain exemplary embodiments, the shell has an average thickness of at least 0.13 mm, for example, between 0.1 mm to 0.5 mm, for example, about 0.25 mm.

The shell thickness may vary based on core or element geometry. In some embodiments, the shell 12 may have a variable thickness. For instance, the shell 12 may have a body thickness, an edge thickness, and/or an end thickness which may each be independently selected. The body portion of the shell may refer to the shell covering a lateral side of the magnetic core, or side 12a as shown in FIG. 1A. The edge portion of the shell may refer to the shell covering an edge portion of the magnetic core, or side 12b as shown in FIG. 1A. The end side of the shell may refer to the shell covering an end portion of the magnetic core, or side 12c as shown in FIG. 1A. The shell 12 thickness may be defined as the dimension between exterior surfaces of sides 12a, 12b, and 12c, as shown in FIG. 1A, relative to an associated core surface of side 11a, 11b, 11c, respectively, as shown in FIG. 1B.

The shell thickness may be selected to enable the plurality of elements to magnetically assemble into a selected conformation, for example, by introducing space between the magnetic core and the ends, edges, and exterior perimeter of the element. In some embodiments, the edge side 12b thickness may be less than the body side 12a thickness. The shell may have an edge side 12b thickness to body side 12a thickness ratio of between 1:1 to 1:5, for example, between 1:1 to 1:2, between 1:2 to 1:3, between 1:3 to 1:4, or between 1:4 to 1:5. In some embodiments, the end side 12c thickness may be greater than the body side 12a thickness. The shell may have an end side 12c thickness to body side 12a thickness ratio of between 10:1 to 1:1, for example, 10:1 to 8:1, 8:1 to 6:1, 6:1 to 4:1, 4:1 to 2:1, or 2:1 to 1:1. In some embodiments, the edge side 12 b thickness may be selected to provide a desired overall profile of the uterine implant elements when deployed in the uterine cavity. For instance, diameter of a triad conformation may be selected by controlling edge side 12b thickness of the elements 10.

The shell may have an edge side 12b thickness (thickness B) of between 0.05 mm to 0.5 mm, for example, 0.05 mm to 0.1 mm, 0.1 mm to 0.15 mm, 0.15 mm to 0.2 mm, 0.2 mm to 0.25 mm, 0.25 mm to 0.3 mm, 0.3 mm to 0.35 mm, 0.35 mm to 0.4 mm, or 0.4 mm to 0.5 mm. The shell may have a body side 12a thickness (thickness A) of 0.1 mm to 1.5 mm, for example, 0.1 mm to 0.15 mm, 0.15 mm to 0.25 mm, 0.25 mm to 0.5 mm, or 0.5 mm to 1.5 mm. The shell may have an end side 12c thickness (thickness C) of between 0.25 mm and 1.5 mm, for example, 0.25 mm to 0.5 mm, 0.5 mm to 1.0 mm, or 1.0 mm to 1.5 mm. In some embodiments, the end portion of the shell may be domed. In such embodiments, the end side 12c thickness may refer to the thickness of the shell at the center of the dome. In other embodiments, the end portion of the shell may be truncated. In such embodiments, the end side 12c thickness may refer to the thickness of the shell at the center of the truncated side.

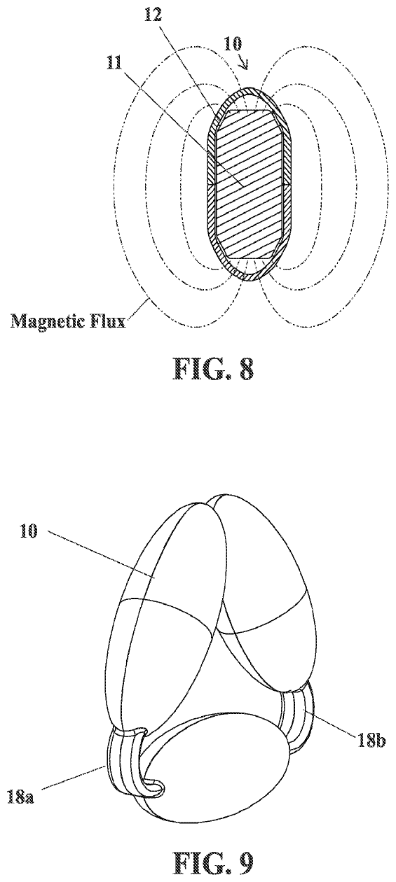

FIG. 8 is a sectional view of an element shown with a diagrammatic representation of magnetic flux patterns, indicative of a magnetically charged inner core 11. As shown in the exemplary embodiment of FIG. 8, the magnetic flux is oriented symmetrically about the center of core 11, typical of an axially charged body. The magnetic flux of an axially charged body flows outward from one end, referred to as a north pole of the magnet, toward and back into the opposite end, referred to as a south pole of the magnet, and through the core in a continuous cycle. The magnetic flux generally flows through the axial end surfaces of the magnetic body, including the peripheral edges. Magnetic flux also generally flows uninterrupted through inert materials. Thus, as shown in the exemplary embodiment of FIG. 8, the magnetic flux flows through inert shell 12 on both ends of the axially charged magnetic core 11.

The elements disclosed herein may have a core comprising a north pole at a first end and a south pole at the opposite end. Typically, north (N) and south(S) poles of magnetically charged bodies will be attracted. A north pole typically repels a north pole. Similarly, a south pole typically repels a south pole. Increasing distance between north and south ends of two opposed magnets will typically reduce the force of magnetic attraction, in a relationship that is generally exponential relative to the increased distance. Conversely, bringing the north and south ends of two opposed magnetically charged bodies closer together (reducing the distance), will typically increase the force of magnetic attraction, in a relationship that is generally exponential relative to the decreased distance. Accordingly, the elements may be designed (for example, have a core and/or shell dimensioned) to have a selected magnetic force between one another when positioned in proximity, encouraging a given number of elements to adopt a desired conformation by magnetic attraction to each other.

Referring to FIG. 1C, the element may have a core and shell dimensioned to self-assemble three elements into a triad conformation when brought into proximity with each other. As shown in FIG. 1C, the exemplary element 10 has the smallest shell thickness on side 12b (thickness B), intermediate thickness on side 12a (thickness A), and largest thickness on side 12c (thickness C, which refers to the thickness of the shell and a void space). It should be understood that edge side 12b and end side 12c generally refer to the sides on both halves of the element 10, however the thicknesses of each side may be independently selected. In some embodiments, as shown in FIG. 1C, the dimension between an exterior surface of side 12a, 12b, and 12c relative to an associated core surface of side 11a, 11b, 11c, respectively, may include a void space and the shell thickness, for example, as shown with respect to thickness C of FIG. 1C. Thus, in some embodiments, the element may include a void space, inert filler, or clearance gap, between the magnetic core 11 and the shell 12 to accommodate a toleranced fit between a physical portion of the shell 12 and the encased internal core 11. The shell and void space, inert filler, and/or clearing gap may form a total thickness (providing a dimension between the exterior surface of the shell and the associated core surface) of between 0.05 mm and 1.50 m, for example, 0.05 mm to 0.1 mm, 0.1 mm to 0.25 mm, 0.25 mm to 0.5 mm, 0.5 mm to 1.0 mm, or 1.0 mm to 1.5 mm.

In one exemplary embodiment, as shown in FIG. 1C, three elements 10, each with axially charged magnetic cores 11, are brought near each other. Opposite poles (N/S) attract to self-arrange the elements. By designing the end side 12c to have the greatest thickness and the edge side 12b to have the smallest thickness, the elements 10 will generally favor self-assembly by joining edges 12b of two elements 10 (“bevel to bevel”). Therefore, by selecting the shell thicknesses, the elements 10 may be designed to favor self-assembly into a selected conformation, such as a triad conformation. The shell thicknesses may also be selected or designed to favor self-assembly of the elements 10 into a generally linear conformation, for example, when confined within a catheter for insertion and/or retrieval.

The shell may be formed of or comprise an inert material. In particular, the shell may be formed of a high coercivity material, for example, a material preventing degradation, e.g., by contact with tissues. The shell may be a polymeric material. The shell may be a non-toxic and physiologically acceptable material, such as Teflon®, silicon, polyethylene, polypropylene, polyether ether ketone (PEEK), or ethylene vinyl acetate (EVA) elastomers. Other shell materials, such as any shell material available to one of ordinary skill in the art suitable for in uterus use, may be used for the elements disclosed herein.

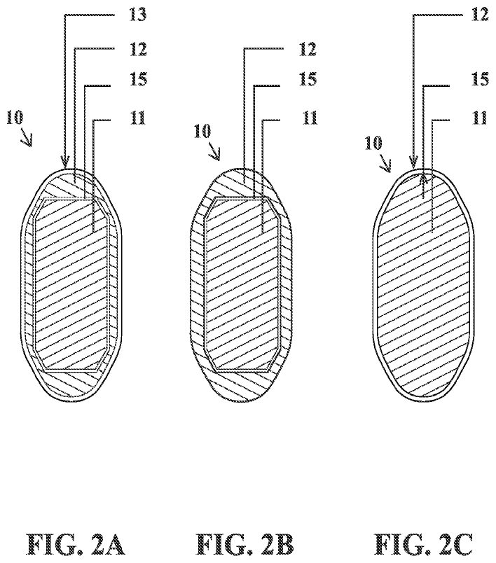

In some embodiments, the element 10 may comprise an adhesive layer 15, as shown in FIGS. 2A-2C. The adhesive layer 15 may be positioned between the magnetic core 11 and the shell 12. The shell 12 may prevent oxidative corrosion of the magnetic core 11. The adhesive layer 15 may be provided to improve surface properties of the core 11. The adhesive layer 15 may have a nominal thickness, for example, about or less than 0.1 mm, between 0.1 mm and 0.01 mm, or less than 0.01 mm.

In the exemplary embodiment of FIG. 2A, the internal magnetic core 11 may be encased within an elliptically shaped shell 12. The coating 13, e.g., anti-fertility agent coating or other coating, may be exterior to the shell 12. In the exemplary embodiment shown in FIG. 2B, the internal magnetic core 11 may be encased within an elliptically shaped shell 12. The anti-fertility agent, if present, may be part of the shell 12 layer. In the exemplary embodiment of FIG. 2C, the magnetic core 11 may be generally elliptical. The magnetic core 11 may be encased within a thin shell 12. As in the embodiment of FIG. 2B, the element 10 of FIG. 2C may also comprise an anti-fertility agent as part of the shell 12.

In some embodiments, the uterine implant element may be free of an anti-fertility agent. In some embodiments, the uterine implant element may be free of a hormonal anti-fertility agent. The element may be free of a cytotoxic anti-fertility agent.

In some embodiments, the element may comprise a coating, e.g., an anti-fertility agent coating. The coating may be an exterior layer 13 of the element 10 (FIG. 2A). The coating may be embedded in the shell 12, for example, as shown in FIGS. 2B-2C. In some embodiments, the anti-fertility agent may be released from the shell 12. In such embodiments, the shell may be formed of an absorbent material, for example having a microstructure, to provide time-controlled release of an anti-fertility agent.

The coating 13, e.g., anti-fertility agent coating or other coating, may be applied, for example, by an electrostatic spray, electroplating, electroless plating, ion deposition, or other method. The coating 13 may be positioned exterior to the magnetic core 11, for example on an exterior surface of the core 11, on an exterior surface of the adhesive 15, or on an exterior surface of the shell 12. The anti-fertility agent coating 13 or anti-fertility agent embedded within and exuding from the shell 12 may be a slow-release agent and/or an extended-release agent. The anti-fertility agent coating 13 and/or shell 12 may be designed to provide a predetermined release profile of the anti-fertility agent. For example, the coating 13 and/or shell 12 may be designed to provide release of a therapeutic amount of the anti-fertility agent over a period of 12, 18, 24, 30, 36, 48, 60, 72, 84, 96, 108, 120, 132, 144 months or more.

The anti-fertility agent may be a hormonal agent, a cytotoxic agent, or a combination thereof. The anti-fertility agent may be a substantially non-released agent. In other embodiments, the anti-fertility agent may be a slow-release agent. Each element may have 400 mm² or less of the anti-fertility agent, for example, 300 mm² or less, 200 mm² or less, 150 mm² or less, 100 mm² or less, 75 mm² or less, or 50 mm² or less. The IUD system (e.g., as formed of more than one element) may have 500 mm² or less of the anti-fertility agent, for example, 400 mm² or less, 300 mm² or less, 200 mm² or less, 150 mm² or less, 100 mm² or less, or 75 mm² or less. The IUD system (e.g., as formed of more than one element) may have 400 mm² to 500 mm², 300 mm² to 400 mm² , 200 mm² to 300 mm² , or 100 mm² to 200 mm² of the anti-fertility agent. In certain exemplary embodiments, for example, for use with a human subject, the total active surface area of the anti-fertility agent may typically range between 150 mm² and 400 mm² , for example, 175 mm² and 380 mm².

In some embodiments, the element is permeable to release of the anti-fertility agent at a low rate. Upon insertion in the uterus, the element may release a therapeutically effective amount of the anti-fertility agent. The therapeutically effective amount may be a contraceptive and/or fertility suppressing amount. The therapeutically effective amount may be an amount effective to provide treatment or prevent a uterine disease, condition, or symptom thereof. The element may include, for example, embedded in the shell or as an independent layer, a therapeutically effective amount of the anti-fertility agent.

The anti-fertility agent may be a hormonal agent. Exemplary contraceptive anti-fertility agents include progesterone or a progestogen, e.g., progestin. The therapeutic amount of the anti-fertility agent in each element may be between 1 mg and 60 mg, e.g., between 1 mg and 3 mg, between 3 mg and 5 mg, between 5 mg and 7 mg, between 7 mg and 10 mg, between 10 mg and 20 mg, between 20 mg and 30 mg, between 30 mg and 40 mg, between 40 mg and 50 mg, or between 50 mg and 60 mg.

In some embodiments, the element is substantially free of a hormonal agent. For instance, in some embodiments, the element is substantially free of progesterone, or progestogen, e.g., progestin.

The anti-fertility agent may be a cytotoxic agent. One exemplary cytotoxic agent is copper. One exemplary cytotoxic agent is nonoxynol 9 (N-9).

The element 10 may contain protrusions, such as rings or ridges 14 (as shown in FIG. 1A), comprising the cytotoxic agent, on an exterior surface of the element 10. The element 10 may contain the cytotoxic agent embedded in the shell 12, which is substantially flush with the surface. The element 10 or shell 12 may contain cytotoxic agent microparticles. At least a portion or the entire outer surface of the element 10 or shell 12 may be or contain the cytotoxic agent.

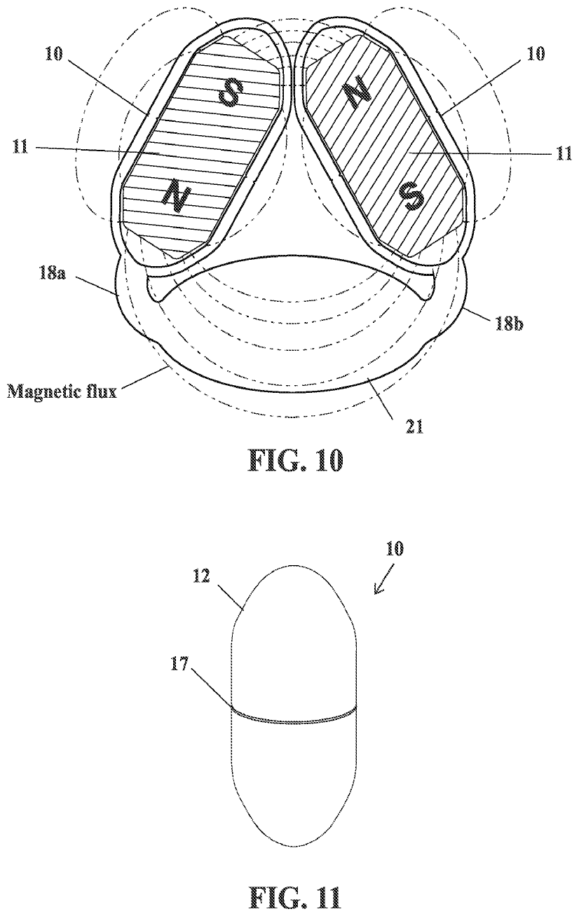

In some embodiments, the element 10 may have a smooth or substantially smooth exterior surface (FIG. 11). The smooth or substantially smooth exterior surface may be an anti-fertility agent exterior surface, e.g., a copper surface. In other embodiments, the element 10 may have one or more surface modification 14, as shown in FIG. 1A. One exemplary surface modification is a protrusion, as described above. Another exemplary surface modification is an indentation. Additionally, or alternatively, the element 10 may have a textured surface. The surface modification 14 may be or comprise, for example, shallow grooves, undulations, indentations, protrusions, articulations, texture, rings, ridges or any other three-dimensional feature.

In certain exemplary embodiments, the protrusion or indentation may be elongated. The surface modification may have a width of between about 0.1 mm and 0.5 mm, for example, between about 0.2 mm and 0.4 mm, or about 0.3 mm. The surface modification 14 may be longitudinal (spanning at least a portion of the length of the body of the element), lateral (spanning at least a portion of the width of the body of the element), or a combination thereof.

The surface modification may comprise or be formed of the anti-fertility agent. The surface modification may be provided to increase surface area of the anti-fertility agent, for example, to increase dosage of the anti-fertility agent.

In one exemplary embodiment, the element 10 may have a smooth exterior copper surface (FIG. 11). The shell 12, which may comprise the anti-fertility agent, for example, a copper shell, may be formed by laser welding a plurality of shell portions together. In the exemplary embodiment of FIG. 11, the shell 12 is formed by laser welding a top shell portion to a bottom shell portion. Thus, the element 10 may comprise a laser weld seam 17. The seam 17 may be vertical, horizontal, or any other orientation. In some embodiments, the element 10 may comprise more than one seam 17. The seam 17 may have a thickness selected to be minimal (produce a negligible reduction in surface area of the shell 12).

In some embodiments only one element 10 is inserted in the uterus of the subject. In other embodiments, more than one element 10 is inserted into the uterus of the subject to form the IUD system. For example, in some embodiments, at least two, three, four, five, six, seven, eight, nine, ten, or more elements 10 may be inserted into the uterus of the subject to form the IUD system. When more than one element 10 is inserted, each element 10 may be smaller (than if only using one), as the multiple elements 10 will self-assemble or arrange themselves into a larger IUD system internally (in uterus) by magnetic force. The north pole of the magnetic core 11 of one element 10 may attract the opposite south pole of the magnetic core 11 of another element 10 to assemble multiple elements 10 together into a larger conformation that resists expulsion from the uterus. Multiple elements 10 may be conformably adjoined, for example, to form a stable conjoined trilobular, circular or coiled conformation. The elements may generally assemble or arrange themselves into a lower energy conformation.

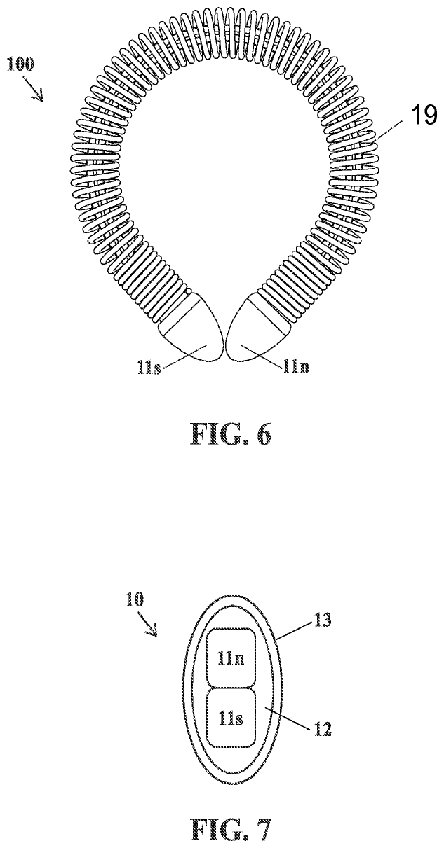

In some embodiments, one element may form the IUD system. One exemplary element 100 having a total length of at least two, three, or more elements 10 (as previously described, for example, at least 3 mm to 30 mm) may be used to form the IUD system (FIG. 6). The element 100 may have a flexible or semi-flexible body. The element 100 may have a magnetic core having a north pole and a south pole as previously described or a plurality of magnetic cores arranged to position a north pole of a first magnetic core and a south pole of a second magnetic core at opposite ends. The north pole may attract the south pole to assemble the element 100 into a conformation that resists expulsion from the uterus, for example, a circular or coiled conformation.

In certain embodiments, the single element IUD system as shown in FIG. 6 may be designed to assemble (for example, into the circular or coiled conformation) by spring-loaded mechanism. The element may be substantially non-magnetic or may comprise a single magnet on one end. The spring-loaded mechanism may be provided by spring 19 extending between opposite ends of the element 100.

Thus, while the disclosure may generally refer to a plurality of elements, it should be understood that a single element having a flexible body being dimensioned similarly, e.g., having a similar width and total length as a plurality of elements, may also be used.

Thus, the IUD system may include an element having a length of 30 mm or less, for example, 3 mm to 30 mm, comprising a magnetic core and a coating exterior to the magnetic core. The magnetic core may comprise at least one of a north pole and a south pole. In some embodiments, the magnetic core may comprise a north pole opposite a south pole. The magnetic core may be formed of a north pole magnetic core and a south pole magnetic core. The magnetic core may be dimensioned to assemble the element into a conformation that resists expulsion from the uterus of a subject.

The one or more elements may generally be independent from one another. For example, the one or more elements may be free of any connective threading or filament.

However, in some embodiments, if more than one element is utilized in the IUD system, the elements may be threaded to each other with a filament (e.g., a monofilament or tail string) or any other connector. In some embodiments, elements which are threaded or connected to each other may still be free of a tail string. Thus, in some embodiments, threading or other connectors may be utilized only between adjacent elements.

FIG. 9 shows an exemplary IUD system having three elements 10, joined by two filaments 18a, 18b, each filament 18a, 18b extending between two adjacent elements 10. A first element and a last element are not joined by a filament. The exemplary elements 10 shown in FIG. 9 do not contain a tail string. The first element and the last element may each include a magnetic core that enables the threaded elements to assemble into a conformation that resists expulsion from the uterus. The magnetic core of the first element may be or comprise a north pole, while the magnetic core of the last element may be or comprise a south pole. The central element (or any element positioned between a first and a last element) may be non-magnetic. In other embodiments, the central element or any element positioned between a first and a last element may also comprise a magnetic core. Any combination of magnetic and non-magnetic elements may be used.

FIG. 10 shows an exemplary IUD system, such as the one shown in FIG. 9, with a diagrammatic representation of magnetic flux patterns. The exemplary IUD system of FIG. 10 includes two magnetic elements 10 at opposite ends of the system with a non-magnetic element 21 positioned between the magnetic elements 10. The magnetic flux of each of the elements 10 of the system of FIG. 10 is oriented symmetrically about the center of each magnetic core 11, as shown in the single axially charged body of FIG. 8. As shown in FIG. 10, an IUD system having two magnetic elements 10 and a non-magnetic element 21 may self-assemble into a triad conformation by magnetic force.

The connective threading or filament may be flexible to enable the elements to assemble into an axial or generally aligned conformation. The connective threading or filament may have a length selected to allow the elements to assemble into a desired conformation, such as a triad conformation. The threading or filament may be fixed to an exterior surface of the elements. In certain embodiments, the connective threading or filament may be integrally molded, e.g., injection molded, with the shell.

In some embodiments, the connector may extend from one element as a longer filament or tail string for retrieval of the IUD system. The filament may generally be formed of or coated with an inert material. The filament material may be selected to provide a desired structural stability in the uterine cavity. Exemplary filament materials include nitinol, e.g., nitinol wire, nylon, e.g., nylon sutures, polypropylene, polyethylene, or polyether, such as polyether ether ketone (PEEK), and others. In some embodiments, the elements and/or IUD system may be substantially free of nitinol.

The magnetic core 11 may be dimensioned to assemble the plurality of elements into a conformation that resists expulsion from a uterus of the subject. The conformation may be the lowest energy conformation for magnetic assembly. In certain exemplary embodiments, the conformation is a triad or triangular conformation.

Each element 10 having a magnetic core 11 may have at least one of a north pole and a south pole, optionally both a north pole and a south pole at opposite ends. The magnetic core 11 may be an axially charged magnetic core (charged about a central axis of the magnetic core). The plurality of elements 10 may assemble by attracting an end of an alternate element 10 having an opposite charge or pole. Furthermore, the plurality of elements 10 may assemble by repelling an end of an alternate element 10 having a like charge or pole. In one exemplary embodiment, the lower energy conformation (for three elements) is a triad conformation (FIGS. 1B, 1C, 1E). In other embodiments, the lower energy conformation (for two or more elements) is a raft conformation. In the raft conformation, the plurality of elements 10 align laterally in a parallel array. The conformation may be flexible and/or variable, e.g., conformable to natural movement of surrounding tissues.

The magnetic core 11 may be, for example, cylindrical, polygonal, e.g., rectangular (e.g., cubed), triangular, pentagonal, hexagonal, or faceted. Thus, the core may have a rounded, circular, oval, polygonal, e.g., rectangular, square, triangular, pentagonal, hexagonal, or faceted cross-section. In certain exemplary embodiments, the magnetic core 11 may have a circular cross section. The circular cross section may be selected to achieve a maximal magnetic force per volume of an element 10, which may be beneficial when providing an element 10 capable of passing through a minimally sized lumen of a cylindrical catheter (of the inserter or retriever). Thus, in certain embodiments, an element 10 having a circular cross section may provide maximal magnetic force while minimizing patient discomfort during insertion or retrieval.

In some embodiments, the core 11 may be oval or ellipsoidal, for example, having a rounded and elongated outline or shape, like that of an elliptical egg.

The core may be formed of one or more magnetic bodies. In some embodiments, the core may be formed of a single magnetic body having a north pole and a south pole, optionally a north pole on one end and a south pole on an opposite end (FIG. 7). In other embodiments, the core may comprise or be formed of two or more magnetic bodies (FIG. 6), each magnetic body having at least one north pole (11n) positioned on one end of the element and at least one south pole (11s) positioned on an opposite end of the element. The two or more magnetic bodies may be positioned such that opposite poles 11n, 11s of the respective magnetic bodies come together to self-assemble the element into a desired conformation. In the exemplary embodiment of FIG. 7, magnetic bodies are positioned on opposite ends of a central axis of the element 100, each magnetic body having an opposing pole 11n, 11s, respectively, on a far end of the magnetic body, such that poles 11n, 11s come together to self-assemble the element 100 into a coiled or circular conformation.

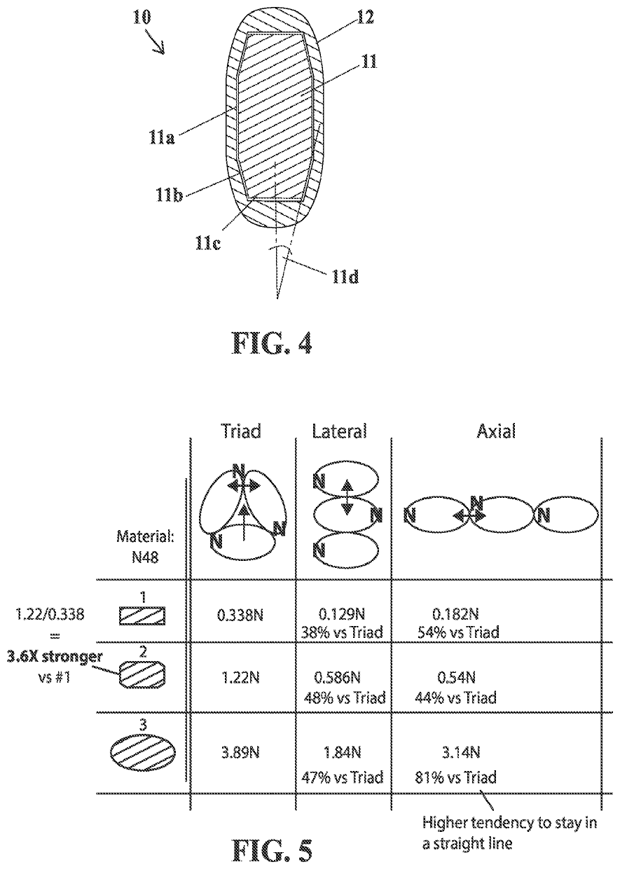

The magnetic core 11 may have generally blunt edges or rounded edges, for example, to form an ellipsoidal structure. In certain embodiments, the magnetic core 11 may have beveled edges 11b to induce self-assembly of three devices into a triad conformation. The beveled edges 11 b may be formed by bevel angle 11d, as shown in FIG. 4. The beveled edge 11b may additionally facilitate inclusion of a larger or maximally sized magnetic core 11 within an elliptically shaped element 10, which may be used to provide a greater magnetic force per volume of the element. Thus, the dimensions of the magnetic core, e.g., length of sides 11a, 11b, 11c and cross-sectional geometry and shell, e.g., thickness of sides 12a, 12b, 12c, (FIGS. 1A-1B) may be selected or designed to induce self-assembly after deployment and maintain a sufficient magnetic force to resist expulsion. In certain embodiments, the dimensions of shell, e.g., thickness of sides 12a, 12b, 12c, may be selected to provide sufficient magnetic force to form a stable IUD system that conforms to the uterine cavity, adapting cyclical changes in response to the natural movement of uterine tissues.

The edge side 11b of the core 11 may be defined as a side of the core 11 that joins the lateral side 11a to the distal end 11c of the core 11, as shown in the sectional view of FIG. 4. The width of the core 11 may be defined as a dimension extending from the lateral side 11a to an opposite lateral side (optionally a diameter of a magnetic core having a circular cross section). The length of the core 11 may be defined as a dimension extending from one distal end 11c to an opposite distal end.

The core 11 may have an edge side 11b on either or both ends. The edge side 11b may extend around a perimeter of the core 11, for example, around a circumference of the cross-section of the core 11. In some embodiments, the edge side 11b may be beveled. A beveled edge may be defined as an edge that forms an angle between the distal end 11c and the edge side 11b. The bevel angle 11d may be defined as an angle formed between a center of end 11c (optionally a center of the circular cross sectional area) and edge side 11b, as shown in FIG. 4. The bevel angle may be from about 5°-50°, for example, from about 5°-15°, 15°-30°, 20°-40°, 30°-45°, or 30°-50°. In certain exemplary embodiments, the bevel angle may be about 30°.

In one exemplary embodiment, as shown in the diagram of FIG. 1D, a 30° bevel angle forms a circumferential 60° inclusive angle forming a conical surface about both ends of a magnetic core 11 having a circular cross section. When a magnetic core 11 is encased within an elliptically shaped shell 12 to form element 10, the tapered end side 12c of the elliptical shell 12 will generally follow the conical surface of the magnetic core 11.

In one exemplary embodiment, when a set of three elements 10, each containing a core 11 with a 30° bevel angle forming a 60° inclusive end angle, are brought together (as shown in FIG. 1E), the conical surfaces of each magnet core 11 are typically induced to self-assemble into a magnetically attracted triad conformation or conformal triangular structure (FIG. 1E). The edge sides 11b of two adjacent cores 11 will typically be separated by the sum of the edge side 12b thicknesses (FIG. 1C). Thus, when the exemplary elements 10 of FIG. 1E are introduced into a uterine cavity, the three magnetic elements 10 are induced to form a magnetically conjoined structure effective to resist expulsion from the uterine cavity.

In some embodiments, the core may be dimensioned to have a lateral side 11a length to edge side 11b length ratio of from about 1:1 to 5:1, for example, 1:1 to 2:1, 2:1 to 3:1, 3:1 to 4:1, or 4:1 to 5:1. The core may have a lateral side 11a length of 0.5 mm to 10 mm, for example, 0.5 mm to 1 mm, 1 mm to 3 mm, 1 mm to 5 mm, or 5 mm to 10 mm. The core may have an edge side 11b length of 0.1 mm to 5 mm, for example, 0.1 mm to 0.5 mm, 0.3 mm to 1.0 mm, 0.5 mm to 1.0 mm, 1.0 mm to 3 mm, or 3 mm to 5 mm. The core may be dimensioned to have a distal end 11c length of about 1 mm to 6 mm, for example, 1 mm to 2 mm, 1 mm to 3 mm, 2 mm to 4 mm, 3 m to 5 mm, or 4 mm to 6 mm.

The magnetic core 11 may form at least 50% of a volume of the element 10. In some embodiments, the magnetic core may form at least 55%, at least 60%, at least 65%, at least 70%, at least 75%, at least 80%, at least 85%, at least 90%, at least 95%, or at least 99% of the volume of the element.

The magnetic core 11 may be dimensioned to provide a selected magnetic force of attraction between two elements, for example, between two elements at opposite poles. The magnetic force may be effective to resist expulsion from the uterus of the subject. The magnetic force may be effective to adapt to the natural movement of the uterine tissues. In some embodiments, the core 11 may be dimensioned to provide about 0.1-22 N of magnetic force, for example, about 0.1-5 N, about 0.1-0.5 N, about 0.5-0.75N, about 0.75-1.0 N, about 1.0-1.25 N, about 1.25-1.5 N, about 1.5-2.0 N, about 2.0-3.0 N, about 3.0-4.0 N, or about 4.0-5.0 N of magnetic force. The dimensionality of the core that may be selected to provide the desired magnetic force includes the length and width (or diameter) of the core, the geometry of the cross-section of the core, and the geometry of the edges of the core, for example, length and/or angle of bevel. Additionally, the material of the core may be selected to provide the desired magnetic force.

Furthermore, the dimensionality of the shell 12 may be selected to provide the desired magnetic force. For instance, the thickness of the shell may be selected to control distance between two adjacent magnetic cores, for example, two cores at opposite poles, or more than two magnetic cores in an IUD system. In some embodiments, the shell 12 may have a thickness selected to position opposing magnetic cores at 0.25 mm-1.0 mm from one another, for example, 0.25 mm-0.5 mm or 0.5 mm-1.0 mm. In certain exemplary embodiments, the thickness of the shell may be selected to position opposing magnetic cores no more than 0.5 mm from one another.

Additionally, the dimensionality of the magnetic core may be selected to provide a selected magnetic force of attraction between an element and a retrieval magnet. The magnetic core may be dimensioned to de-assemble to plurality of elements from the conformation that resists expulsion to a conformation that allows retrieval. The conformation that allows retrieval may be, for example, an axial or generally aligned conformation. The magnetic core may be dimensioned to de-assemble into a generally linearly connected conformation, e.g., the axial conformation or a linear train-like conformation, when brought into close proximity with a magnetic retrieval device.

In certain embodiments, 1-4 elements are used to form the IUD system. The size and number can be dependent on the species (human or non-human mammal). For example, when multiple elements are used, the size of each element may be individually about 2 mm to about 30 mm in length, for example, about 2 mm to about 15 mm in length or about 2 mm to about 10 mm in length, and about 2 mm to about 15 mm in width, for example, about 2 mm to about 10 mm in width or about 2 mm to about 6 mm in width or about 2 mm to about 4 mm in width. The size may be selected to allow the placement of the elements in the uterus at any stage of the menstrual cycle, while still retaining a high retention rate. The retention rate of the IUD system may be at least about 90%, at least about 95%, at least about 99%, or at least about 100% retention rate. The size may be selected to allow insertion of the elements through the cervix of the subject, for example, easier and less painful insertion into the uterus of the subject.

In some embodiments, the health, pregnancy/non-pregnancy status, and/or reproductive system dimensionality of the subject may be determined before insertion of the IUD system. For instance, an ultrasound of the uterus may be performed in two dimensions or three dimensions to determine the health, pregnancy/non-pregnancy status, and/or dimensions to accept or reject the subject for use of the IUD system. In general, the minimum precaution may be to establish non-pregnancy of the subject before introducing the IUD system. In some embodiments, the health, pregnancy/non-pregnancy status, and/or dimensionality of the reproductive system of the subject may be determined to aid in the choice of size and/or number of elements to be used. The dimensionality of the reproductive system of the subject may include, for example, anatomical geometry, anatomical dimensions, and/or anatomical positioning (e.g., normal or abnormal positioning of the uterus).

The methods may comprise delivering to a subject a plurality of uterine implant elements using a delivery device (also referred to as an “inserter” or “introducer” or “applicator” herein) dimensioned to allow placement of the plurality of elements in the uterus of the subject. In one particular embodiment, the delivery device may be specially designed to accompany the IUD elements described herein. In some embodiments, the inserter may comprise a catheter dimensioned to deliver the elements to the uterine cavity of the subject through the cervical canal.

The methods may comprise inserting the inserter through the cervical canal of the subject and depositing the implant elements in the uterine cavity of the subject before removing the inserter from the subject. The delivery device may be dimensioned for use with a target subject. For example, the delivery device may be dimensioned for use with a human subject. Self or assisted insertion may be performed, for example, insertion conducted by a trained practitioner and/or medical professional. The catheter may be dimensioned to deliver the elements to the uterine cavity of the subject without the use of a balloon, for example, a dilating balloon. Thus, in some embodiments, the inserter may be free of any expandable or inflatable component, such as a balloon.

The methods may comprise delivering the plurality of elements individually. For instance, the inserter may be dimensioned to deliver the plurality of elements individually. In other embodiments, the methods may comprise delivering the plurality of elements sequentially or simultaneously. For instance, the inserter may be dimensioned to deliver the plurality of elements simultaneously, optionally in a sequential (axial or generally aligned) arrangement. In certain embodiments, the inserter may comprise a catheter dimensioned to deliver the plurality of elements in a sequential, axial, or generally aligned arrangement.

The methods may comprise retrieving the plurality of uterine implant elements from the subject using a retrieval device (also referred to as a “retriever” herein) dimensioned to retrieve the plurality of elements from the uterus of the subject. In one particular embodiment, the retrieval device may be specially designed to accompany the IUD elements described herein.

The methods may comprise inserting the retriever through the cervical canal of the subject to reach the uterine cavity of the subject, collecting at least one element, and optionally the plurality of elements simultaneously, and removing the retriever with the elements from the subject. The retrieval device may be dimensioned for use with a target subject. For example, the retrieval device may be dimensioned for use with a human subject. Self or assisted retrieval may be performed. The catheter may be dimensioned to retrieve the elements from the uterine cavity of the subject without the use of a balloon, for example, a dilating balloon. Thus, in some embodiments, the retriever may be free of any expandable or inflatable component, such as a balloon.

The retriever may generally include a magnetic end effective to attract the elements. Upon bringing the magnetic end into close proximity with the elements, the plurality of elements may be induced to de-assemble into an axial or generally aligned conformation that allows retrieval from the uterus of the subject with the retrieval device. The magnetic end may be positioned on a distal end of a wand or elongated structure dimensioned to retrieve the plurality of elements from the uterus of the subject. In one particular embodiment, the retriever may be similar in dimensions and assembly as the inserter. For instance, in some embodiments, the retriever may comprise a catheter having a magnetic end dimensioned to retrieve the plurality of elements in sequential, axial, or generally aligned arrangement.

The methods may comprise retrieving the plurality of elements individually. For instance, the retriever may be dimensioned to retrieve the plurality of elements individually. In other embodiments, the methods may comprise retrieving the plurality of elements sequentially or simultaneously. For instance, the retriever may be dimensioned to retrieve the plurality of elements simultaneously, optionally in a sequential (axial or aligned) arrangement.

In certain embodiments, retrieval may also be carried out by a filament or tail, e.g., a length of filament or string connected to and/or interconnecting the plurality of elements (similar to removal of conventional contraceptive devices), optionally threading the devices together. However, in other embodiments, the elements are free of a tail string and/or any filament or string.

The IUD elements 10 disclosed herein may be administered, e.g., delivered, to the uterus of the subject and/or retrieved from the uterus of the subject with reduced pain. Subjects may be asked to rate pain score under the numeric rating scale (NRS) after administration or retrieval of the IUD elements 10. The NRS is a verbal or written determination of a pain level on a scale from 0 to 10, in which 0 represents no pain and 10 represents extreme pain. In some embodiments, an average or maximum NRS pain score for administration, e.g., delivery, of the IUD elements 10 may be less than 10, e.g., less than 9, less than 8, less than 7, less than 6, less than 5, less than 4, less than 3, less than 2, or less than 1. In some embodiments, an average or maximum NRS pain score for retrieval of the IUD elements 10 may be less than 10, e.g., less than 9, less than 8, less than 7, less than 6, less than 5, less than 4, less than 3, less than 2, or less than 1.

In other embodiments, subjects may be asked to rate pain score under the visual analog scale (VAS) after administration or retrieval of the IUD elements 10. The VAS is a graphic rating scale that may utilize a line or a series of graphical depictions, such as cartoon faces, for the subject to select, indicating degrees of pain level. For a line depiction, a first end of the line may represent no pain, with an opposite end of the line representing extreme pain. Subjects may be asked to indicate pain level by identifying a point on the line. In some embodiments, an average or maximum VAS pain score for administration, e.g., delivery, of the IUD elements 10 may be less than 100% across the line (e.g., less than an extreme end point of the line), for example, less than 80% across the line, less than 75% across the line, less than 60% across the line, less than 50% across the line, less than 40% across the line, less than 25% across the line, or less than 20% across the line. In some embodiments, an average or maximum VAS pain score for retrieval of the IUD elements 10 may be less than 100% across the line, for example, less than 80% across the line, less than 75% across the line, less than 60% across the line, less than 50% across the line, less than 40% across the line, less than 25% across the line, or less than 20% across the line.

For the graphical depiction, five graphics may visually represent increasing pain level from no pain to extreme pain. Subjects may be asked to indicate pain level by identifying a specific graphic that demonstrates their pain level. In some embodiments, an average or maximum VAS pain score for administration, e.g., delivery, of the IUD elements 10 may be less than the graphic used to identify extreme pain (e.g., less than the fifth graphic), less than the fourth graphic, less than the third graphic, or less than the second graphic. In some embodiments, an average or maximum VAS pain score for retrieval of the IUD elements 10 may be less than the fifth graphic, less than the fourth graphic, less than the third graphic, or less than the second graphic.

In accordance with one aspect, the disclosure includes methods of providing controlled contraception to a subject and/or methods of facilitating controlled contraception. The methods may comprise delivering to the uterus of the subject a plurality of uterine implant elements. The plurality of elements may be delivered individually or simultaneously. Individual delivery may include, for example, delivery of a single element by a single actuation of a delivery device. Simultaneous delivery may include, for example, delivery of the plurality of elements by a single actuation of a delivery device.

In accordance with one aspect, the disclosure includes methods of treating or preventing a uterine disease, condition, or symptom thereof in a subject and/or methods of facilitating treatment or prevention of a uterine disease, condition, or symptom thereof. The methods may comprise delivering to the uterus of the subject a plurality of uterine implant elements. The plurality of elements may be delivered individually or simultaneously. Individual delivery may include, for example, delivery of a single element by a single actuation of a delivery device. Simultaneous delivery may include, for example, delivery of the plurality of elements by a single actuation of a delivery device.

### Detection Sensor

The methods may comprise scanning the subject for detection of the IUD system after insertion or retrieval. Detection of the elements may be performed by ultrasound or using a detection sensor. Exemplary detection sensors include a magnetic field detector, Gauss meter, or metal detectors.

The detection sensor may provide contactless detection. The detection sensor may indicate detection of the device with a visual and/or auditory alarm and/or by transmitting a notification to a computer or mobile device. The detection sensor may be equipped to detect the device from a distance of 2 inches or more, for example, from at least 2 inches, 4 inches, 6 inches, 8 inches, or 10 inches to 1 ft.

### EXAMPLES

The function and advantages of these and other embodiments can be better understood from the following examples. These examples are intended to be illustrative in nature and are not considered to be limiting the scope of the invention.

### Example 1: Magnetic Force as a Function of Magnetic Core Geometry

Digital simulation studies were performed to evaluate the relative effectiveness of alternative magnetic core geometries to assemble into varied configurations. Magnetic force is a function of magnetic material, core geometry, size, and assembly configuration. Three magnetic core geometries were tested, including: a cylindrical core (1), a core with beveled edges having a circular cross-section (2), and a solid ellipsoidal magnet element (without a shell).

Each configuration was evaluated for magnetic force when assembled into: a triad configuration, a laterally arrayed configuration (magnets aligned side to side), and an axially arrayed configuration (magnets aligned end to end). It should be noted that configurations (1) and (2) were sized to fit within an ellipsoidal shell sized as (3), i.e., a maximal size to be delivered through a particular sized catheter lumen. The data are shown in the table of FIG. 5. As shown in FIG. 5, the elliptical magnet (3) had the greatest magnetic force in each of the three configurations but had a higher tendency to stay assembled in a series configuration (when inserted in the uterus of a subject). The cylindrical core (1) prefers to assemble into a triad conformation, as the magnetic forces in that conformation are stronger than in a lateral or axially arrayed configuration. The core with beveled edges (2) also prefers to assemble into the triad conformation and exhibits much stronger magnetic forces holding the elements together, which is preferred. The cylindrical core (1) had the weakest magnetic force in each of the configurations.

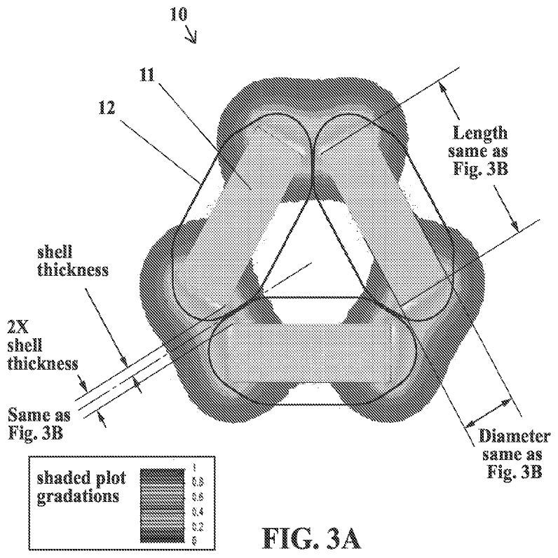

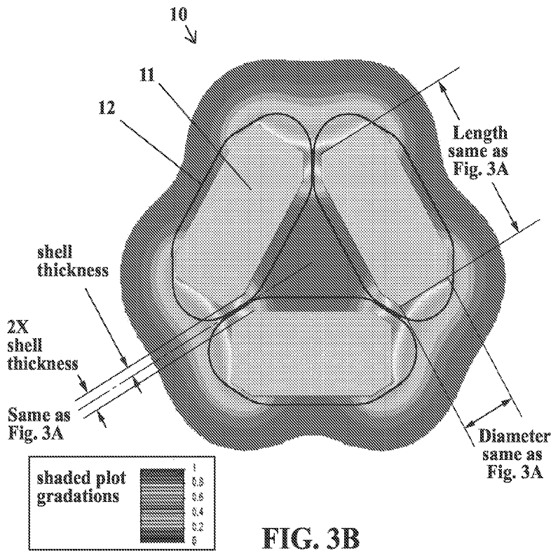

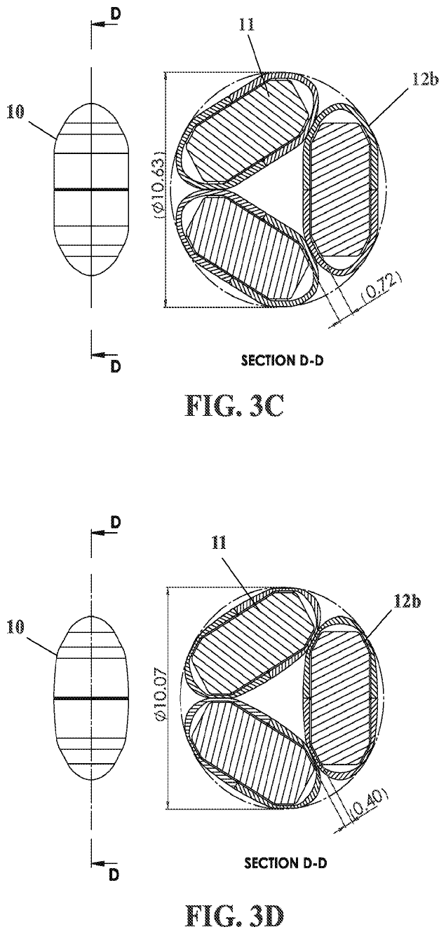

FIGS. 3A and 3B show shaded plot diagrams, prepared to visualize the relative attractive forces of: a cylindrical core (1) (FIG. 3A), as compared to a larger diameter core having a circular cross section and beveled ends (2) (FIG. 3B). Both core geometries are the same length. Both core geometries have the same diameter at each end. The cylindrical core (1) has the weakest magnetic force in each of the configurations. The core with beveled ends (2) exhibited a significantly stronger attractive force in a triad configuration (3.6×) than the cylindrical core (1). Shaded plot gradations, ranging from red (strongest)>yellow>green>cyan>blue (weakest) indicate a relative range of magnitude for attractive force. The red shading (1.0) is barely present in the cylindrical core (1) diagram (FIG. 3A).

FIGS. 3C and 3D show overall geometry of three elements 10 arranged in a triad configuration. The exemplary elements 10 of FIGS. 3C and 3D have cylindrical magnetic cores 11 with beveled edges. The elements 10 of FIG. 3C have a smaller edge thickness 12b (thickness B) than the elements 10 of FIG. 3D. As shown in FIGS. 3C-3D, by reducing edge thickness 12b from 0.36 mm (FIG. 3C) to 0.2 mm (FIG. 3D), the diameter of the overall triad configuration can be reduced from 10.63 mm (FIG. 3C) to 10.07 mm (FIG. 3D).

Accordingly, the elements 10 having a smaller edge thickness 12b self-arrange into a triad configuration having a smaller overall profile and a stronger magnetic attraction between elements 10.

### Example 2: Prophetic Example of a Method of Providing Contraception

A frameless IUD system as disclosed herein will be used to provide contraception to a subject. In particular, three uterine implant elements which make up the IUD system will be delivered to the uterus of a subject with an accompanying handler (developed by 3Daughters, Inc., Fort Lauderdale, FL) to provide contraception. The uterine implant elements will have a magnetic core dimensioned to assemble into a conformation that adapts to the uterine environment and resists expulsion. In this example, the uterine implant elements will have a magnetic core dimensioned to adopt a stable triad conformation in the uterus of the subject. The uterine implant elements will have the following dimensions:
a length of less than 10 mm, for example, between 2.0 mm and 10 mm and a width of between 2.0 mm and 6.0 mm, for example, dimensioned to fit within a 4.0 mm or 4.4 mm OD catheter;
the magnetic core of the uterine implant element will have a rounded cross-sectional area and beveled edges; and
an anti-fertility agent coating forming a shell protectively encasing the magnetic core will have a varied thickness.

The element will generally be ellipsoidal in shape, as provided by the shell. The shell will be dimensioned to enable the three elements to self-assemble into the stable triad configuration upon administration and de-assemble into an axially arrayed (aligned) configuration for retrieval.

Traditional contraceptive devices with a frame (plastic (polyethylene)) T-shaped design cause painful insertions, incidences of perforation of the uterus, expulsion from the uterus, and include required strings for removal from the uterus.

The frameless IUD system disclosed herein will include a plurality of uterine implant elements that conform and adapt to the uterine environment, provide an easier insertion process (with fewer steps, reducing or eliminating pain points), have smooth exterior dimensions, and are structurally independent from each other. Accordingly, the frameless IUD system disclosed herein will provide contraception to a subject with reduced insertion pain, reduced (or eliminated) risk of expulsion (adapting to the uterine environment), reduced (or eliminated) risk of uterine perforation, and without requiring strings for removal.

### Example 3: Prophetic Example of a Method of Providing Treatment or Prevention of a Uterine Disease, Condition, or Symptom Thereof

A frameless IUD system as disclosed herein will be used to provide treatment or prevention of a uterine disease, condition, or symptom thereof (such as abnormal uterine bleeding) to a subject. In particular, three uterine implant elements which make up the IUD system will be delivered to the uterus of a subject with an accompanying handler (developed by 3Daughters, Inc., Fort Lauderdale, FL) to provide treatment or prevention. The uterine implant elements will have a magnetic core dimensioned to assemble into a conformation that adapts to the uterine environment and resists expulsion. In this example, the uterine implant elements will have a magnetic core dimensioned to adopt a stable triad conformation in the uterus of the subject.

The uterine implant elements will have the following dimensions:
a length of less than 10 mm, for example, between 2.0 mm and 10 mm and a width of between 2.0 mm and 6.0 mm, for example, dimensioned to fit within a 4.0 mm or 4.4 mm OD catheter;
the magnetic core of the uterine implant element will have a rounded cross-sectional area and beveled edges; and
a coating forming a shell (optionally a shell of an inert material or a shell of an anti-fertility agent) protectively encasing the magnetic core will have a varied thickness.

The element will generally be ellipsoidal in shape, as provided by the shell. The shell will be dimensioned to enable the three elements to self-assemble into the stable triad configuration upon administration and de-assemble into an axially arrayed (aligned) configuration for retrieval.

Traditional intrauterine devices with a frame (plastic (polyethylene)) T-shaped design cause painful insertions, incidences of perforation of the uterus, expulsion from the uterus, and include required strings for removal from the uterus.

The frameless IUD system disclosed herein will include a plurality of uterine implant elements that conform and adapt to the uterine environment, provide an easier insertion process (with fewer steps, reducing or eliminating pain points), have smooth exterior dimensions, and are structurally independent from each other. Accordingly, the frameless IUD system disclosed herein will provide treatment or prevention of a uterine disease, condition, or symptom thereof to a subject with reduced insertion pain, reduced (or eliminated) risk of expulsion (adapting to the uterine environment), reduced (or eliminated) risk of uterine perforation, and without requiring strings for removal.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only.

Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

1. An intrauterine device (IUD) system including a plurality of elements, each element having a length of 10 mm or less, comprising a magnetic core and a coating exterior to the magnetic core, the magnetic core being dimensioned to assemble the plurality of elements into a conformation that resists expulsion from a uterus of a subject.

2. The IUD system of claim 1, wherein the IUD system includes at least three elements.

3. The IUD system of claim 2, wherein the IUD system includes three elements, each element having the magnetic core dimensioned to assemble the plurality of elements into a triad conformation.

4. The IUD system of claim 1, wherein each element comprises a shell exterior to the magnetic core.

5. The IUD system of claim 4, wherein the IUD system includes three elements, each element having the shell dimensioned to assemble the plurality of elements into a triad conformation.

6. The IUD system of claim 4, wherein the shell has an average thickness between 0.10 mm and 1.50 mm.

7. The IUD system of claim 4, wherein the coating is an anti-fertility agent coating and the anti-fertility agent coating is embedded in the shell.

8. The IUD system of claim 1, wherein the magnetic core is an axially charged magnetic core.

9. The IUD system of claim 8, wherein the magnetic core is dimensioned to de-assemble the plurality of elements into an axial or generally aligned conformation that allows retrieval from the uterus of the subject with a magnetic retrieval device.

10. The IUD system of claim 1, wherein each element consists of the magnetic core and the coating.

11. The IUD system of claim 1, wherein each element has a rounded cross-sectional area having a diameter between 2.0 mm and 6.0 mm.

12. The IUD system of claim 11, wherein each element is dimensioned to pass through the cervix of the subject.

13. The IUD system of claim 12, wherein each element is dimensioned to fit within a 4.0 mm to 5.0 mm OD catheter.

14. The IUD system of claim 1, wherein each element has a length between 2.0 mm and 10 mm.

15. The IUD system of claim 1, wherein the magnetic core has rounded or beveled edges.

16. The IUD system of claim 1, wherein the coating is an anti-fertility agent coating and the anti-fertility agent comprises a cytotoxic agent.

17. The IUD system of claim 16, wherein the cytotoxic agent comprises copper.

18. The IUD system of claim 17, wherein the anti-fertility agent coating has a copper surface area of between 175 mm² and 380 mm².

19. The IUD system of claim 1, wherein the coating is an anti-fertility agent coating and the anti-fertility agent comprises a hormonal agent.

20. The IUD system of claim 1, wherein the coating is substantially free of a hormonal agent.

21. The IUD system of claim 1, wherein the coating is substantially free of nickel and/or nitinol.

22. The IUD system of claim 1, wherein the magnetic core includes a south pole and a north pole opposite the south pole.

23. The IUD system of claim 1, wherein the magnetic core forms at least 50% of a volume of each element.

24. The IUD system of claim 1, wherein each of the plurality of elements are structurally independent from one another.

25. A method of providing controlled contraception or treating or preventing a uterine disease, condition, or symptom thereof in a subject, comprising:
delivering to a uterus of the subject a plurality of elements, each element having a length of 10 mm or less, comprising a magnetic core and a coating exterior to the magnetic core, the magnetic core being dimensioned to assemble the plurality of elements into a conformation that resists expulsion from the uterus of the subject.

26. The method of claim 25, comprising delivering each element individually.

27. The method of claim 25, comprising delivering the plurality of elements sequentially and/or simultaneously.

28. A method of facilitating controlled contraception or treating or preventing a uterine disease, condition, or symptom thereof in a subject, comprising:
providing a plurality of elements, each element having a length of 10 mm or less, comprising a magnetic core and a coating exterior to the magnetic core, the magnetic core being dimensioned to assemble the plurality of elements into a conformation that resists expulsion from the uterus of a subject; and
providing instructions to deliver the plurality of elements to the uterus of the subject.

29. A kit comprising:
an intrauterine device (IUD) system including a plurality of elements, each element having a length of 10 mm or less, comprising a magnetic core and a coating exterior to the magnetic core;
a delivery device dimensioned to allow placement of the plurality of elements in the uterus of a subject; and
instructions to deliver the plurality of elements to the uterus of the subject using the delivery device.

30. The kit of claim 29, further comprising a retrieval device dimensioned to allow retrieval of the plurality of elements from the uterus of the subject.
